(19) United States
(12) Patent Application Publication
(10) Pub. No.: US 2026/0199281 A1
(43) Pub. Date: Jul. 16, 2026

(54) NEW USE OF SIVELESTAT

(71) Applicant: SHANGHAI HUILUN PHARMACEUTICAL CO., LTD., Shanghai (CN)

(72) Inventors: Wenhua LI, Shanghai (CN); Shuisong CAO, Shanghai (CN)

(21) Appl. No.: 19/111,881

(22) PCT Filed: Jul. 6, 2023
(86) PCT No.: PCT/CN2023/105999

§ 371 Date: Mar. 14, 2025

(30) Foreign Application Priority Data
Jul. 7, 2022 (CN) 202210802627.3

(51) Int. Cl. A61K 31/24; A61K 31/42; A61K 31/635; A61P 29/00

(52) U.S. Cl. CPC A61K 31/24; A61K 31/42; A61K 31/635; A61P 29/00

(57) ABSTRACT

The present invention relates to new use of sivelestat, and use of sivelestat alone or in combination with a COX-2 inhibitor in preparation of a drug for treating or alleviating pain, in particular post-traumatic pain, post-operative pain, and inflammatory pain. By means of the combination of the sivelestat and the COX-2 inhibitor, the treatment effect on pain of the COX-2 inhibitor can be significantly improved, the treatment time thereof is significantly prolonged, and the dosage of the drug is reduced.

### TECHNICAL FIELD

The present invention belongs to the field of medicine, and specifically relates to a new therapeutic use of sivelestat or a salt thereof alone or in combination with a COX-2 inhibitor for treating/alleviating pain.

### BACKGROUND

The World Health Organization (WHO) and the International Association for the Study of Pain (IASP) define pain as: pain is an unpleasant sensory and emotional experience associated with actual or potential tissue damage. Pain is a highly complex, heterogeneous and dynamic process involving ascending and descending pathways in the spinal cord, as well as multiple interconnected neurotransmitters and neuromodulatory systems at supraspinal sites. As an important physiological function, pain constitutes the body's self-protection mechanism.

Pain is divided into acute pain and chronic pain. Acute pain mainly involves post-traumatic and post-operative pain, while chronic pain involves a variety of diseases, including nociceptive pain (such as pain caused by osteoporosis and rheumatoid arthritis), neuropathic pain (central, such as post-stroke pain, spinal cord injury, migraine, AIDS-related neuralgia, etc.; peripheral, such as post-herpetic neuralgia, diabetic neuralgia), organ-related pain (such as pain induced by pancreatitis, inflammatory bowel syndrome, etc.), mixed pain (such as low back pain, cancer pain, fibromyalgia, etc.), etc. When chronic pain does not respond to treatment, the cause of the pain cannot be removed, or the pain cannot be treated otherwise, this condition is considered intractable pain. Intractable pain can be a comorbid condition of cancer, severe burns, arthritis, migraine, lower back pain, myocardial ischemia, renal colic or gout.

According to the classification by the site of onset, pain can be divided into three types: somatic pain, visceral pain, and non-specific pain. According to the pathophysiological classification, pain can be classified into nociceptive pain and neuropathic pain.

Neuropathic pain is usually caused by alterations in the pain conduction pathways of the central or peripheral nervous system. The occurrence of neuropathic pain is related to polygenic origins, and multi-cellular and molecular targets, making it difficult to diagnose the exact cause of persistent pain symptoms. Neuropathic pain is mainly attributed to abnormal activity of neurons or immune cells. For example, this type of pain is usually chronic, due to it is caused by some diseases, such as diabetes, nerve damage from surgical operations or trauma.

Commonly used analgesics include non-steroidal anti-inflammatory analgesics (such as aspirin, ibuprofen, rofecoxib, celecoxib, etc.) and opioid central analgesics (such as morphine, pethidine, tramadol, oxycodone, etc.). In addition, some antiepileptic drugs, antidepressants, and selective COX-2 receptor inhibitors are also used in the treatment of neuropathic pain and inflammatory pain. Non-steroidal analgesics have relatively good therapeutic effects on dull pain such as headache, toothache, neuralgia, and muscle and joint pain, but they are almost ineffective on severe pain caused by trauma and colic of internal organs and visceral smooth muscle, and their analgesic intensity is inferior to that of opioids.

Sivelestat is a selective neutrophil elastase (NE) inhibitor and the first drug approved for the treatment of acute lung injury accompanied by systemic inflammatory response syndrome in the world. The daily dosage for treating this disease is 4.8 mg/kg/day (intravenous administration at 0.2 mg/kg/h for 24 hours). Based on its highly selective inhibitory effect on NE, sivelestat is expected to be used in the treatment of many types of diseases.

It is documented in Document 1 that sivelestat sodium can be used for neuropathic pain or chronic pain including neuropathic pain. Specifically, the above pain includes metabolic and hereditary neuropathic pain, diabetic neuropathic pain (DPN), cancerous neuropathic pain, etc. It is also described in Document 2 that prophylactic inhibition of neutrophil elastase can prevent the occurrence of chronic neuropathic pain in mice with osteoarthritis.

The pain therapeutic use of sivelestat sodium disclosed in the prior art is mainly related to neuropathic pain. It has not been reported that sivelestat sodium can be used to treat or alleviate other types of pain, particularly post-traumatic or post-operative pain.
Document 1 WO2016050835 A2
Document 2 Muley et al. Journal of Neuroinflammation (2017) 14:168

### SUMMARY

In a first aspect of the present invention, use of sivelestat or a pharmaceutically acceptable salt thereof in preparation of a medicament for treating or alleviating pain is provided. The pain in the present invention includes acute pain or chronic pain; the acute pain includes post-traumatic pain, post-operative pain, etc.; and the chronic pain includes inflammatory pain.

The present invention further provides use of sivelestat or a pharmaceutically acceptable salt thereof in preparation of a medicament for treating or alleviating post-traumatic pain or post-operative pain.

The present invention further provides use of sivelestat sodium in preparation of a medicament for treating or alleviating post-traumatic pain or post-operative pain.

In a second aspect of the present invention, use of sivelestat or a pharmaceutically acceptable salt thereof in combination with a COX-2 inhibitor in preparation of a medicament for treating or alleviating pain is provided. The pain includes acute pain or chronic pain; the acute pain includes post-traumatic pain, post-operative pain, etc.; and the chronic pain includes inflammatory pain.

The present invention further provides use of sivelestat or a pharmaceutically acceptable salt thereof in combination with a COX-2 inhibitor in preparation of a medicament for treating or alleviating post-traumatic pain or post-operative pain.

The COX-2 inhibitor in the present invention includes, but is not limited to: parecoxib, celecoxib, etoricoxib, valdecoxib, imrecoxib, lumiracoxib, zaltoprofen, meloxicam, diclofenac potassium, lornoxicam, ketorolac tromethamine, nimesulide, etodolac, polmacoxib, etc. In a preferred embodiment of the present invention, the COX-2 inhibitor is parecoxib; and in another preferred embodiment of the present invention, the COX-2 inhibitor is celecoxib.

A dosage of the sivelestat or the pharmaceutically acceptable salt thereof in the present invention is 1.0-300 mg/kg/day.

A weight ratio of the sivelestat or the pharmaceutically acceptable salt thereof to the COX-2 inhibitor in the present invention is from 100:1 to 1:1.

In a third aspect of the present invention, use of sivelestat or a pharmaceutically acceptable salt thereof in combination with parecoxib or celecoxib in preparation of a medicament for treating or alleviating pain is provided. The pain includes acute pain or chronic pain; the acute pain includes post-traumatic pain, post-operative pain, etc.; and the chronic pain includes inflammatory pain.

The present invention also further provides use of sivelestat sodium in combination with parecoxib or celecoxib in preparation of a medicament for treating or alleviating post-traumatic pain or post-operative pain.

The present invention also further provides use of sivelestat sodium in combination with parecoxib in preparation of a medicament for treating or alleviating post-traumatic pain or post-operative pain.

In a fourth aspect of the present invention, a combination product is provided, including components of:
(1) sivelestat or a pharmaceutically acceptable salt thereof; and
(2) a COX-2 inhibitor.

In the combination product in the present invention, a weight ratio of the sivelestat or the pharmaceutically acceptable salt thereof to the COX-2 inhibitor is from 100:1 to 1:1.

In the combination product in the present invention, a dosage of the sivelestat or the pharmaceutically acceptable salt thereof is 1.0-300 mg/kg/day.

The COX-2 inhibitor includes, but is not limited to: parecoxib, celecoxib, etoricoxib, valdecoxib, imrecoxib, lumiracoxib, zaltoprofen, meloxicam, diclofenac potassium, lornoxicam, ketorolac tromethamine, nimesulide, etodolac, polmacoxib, etc.

In a fifth aspect of the present invention, a combination product is provided, including components of:
(1) sivelestat sodium; and
(2) a COX-2 inhibitor, optionally selected from: parecoxib or celecoxib.

In a sixth aspect of the present invention, use of a combination product in preparation of a medicament for treating or alleviating pain is provided, the combination product including components of:
(1) sivelestat or a pharmaceutically acceptable salt thereof; and
(2) a COX-2 inhibitor, optionally selected from: parecoxib or celecoxib.

The pain includes acute pain or chronic pain; the acute pain includes post-traumatic pain, post-operative pain, etc.; and the chronic pain includes inflammatory pain.

The present invention further provides use of a combination product in preparation of a medicament for treating or alleviating post-traumatic pain or post-operative pain, the combination product including components of:
(1) sivelestat or a pharmaceutically acceptable salt thereof; and
(2) a COX-2 inhibitor, optionally selected from: parecoxib or celecoxib.

In a seventh aspect of the present invention, use of a combination product in preparation of a medicament for treating or alleviating pain is provided, the combination product including components of:
(1) sivelestat sodium; and
(2) parecoxib.

The pain includes acute pain or chronic pain; the acute pain includes post-traumatic pain, post-operative pain, etc.; and the chronic pain includes inflammatory pain.

In an eighth aspect of the present invention, a pharmaceutical kit is provided, including the combination product, the product including components of:
(1) sivelestat or a pharmaceutically acceptable salt thereof; and
(2) a COX-2 inhibitor.

The sivelestat in the present invention can be used to treat or alleviate pain, particularly post-operative pain and post-traumatic pain. Further, by combining the sivelestat with the COX-2 inhibitor, particularly with parecoxib or celecoxib in the present invention, it is unexpectedly found that pain can be significantly improved or alleviated, particularly post-traumatic and post-operative pain, the therapeutic dose of COX-2 can be significantly reduced, the side effects caused by using the COX-2 inhibitor are decreased, and the treatment time of the medicament for pain is significantly prolonged.

### BRIEF DESCRIPTION OF THE DRAWINGS

FIG. 1 shows baseline values before drug administration. In FIG. 1, the left bar graphs show mean values, and the right graphs show specific values of 8 animals in each group.

FIG. 2 shows mean values of compounds inhibiting mechanical hyperalgesia induced by rat models of post-operative pain. From left to right, the bar graphs represent a vehicle group, a parecoxib 20 mg/kg group, a sivelestat sodium 10 mg/kg group, a sivelestat sodium 30 mg/kg group, a sivelestat sodium 100 mg/kg group, a sivelestat sodium 30 mg/kg+parecoxib 10 mg/kg group, and a sivelestat sodium 100 mg/kg+parecoxib 10 mg/kg group, respectively.

FIG. 3 shows a specific data distribution of compounds inhibiting mechanical hyperalgesia induced by rat models of post-operative pain.

### DETAILED DESCRIPTION

### Explanation of Terms

The “sivelestat” in the present invention has the following structure:

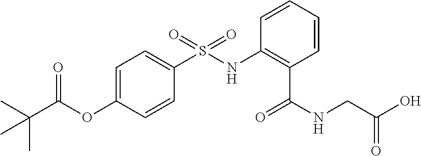 [SMILES: CC(C)(C)C(=O)Oc1ccc(S(=O)(=O)Nc2ccccc2C(=O)NCC(=O)O)cc1]

The “pharmaceutically acceptable salt” can be a sodium salt, a potassium salt, a calcium salt, etc. Preferably, the pharmaceutically acceptable salt is a sodium salt. As a specific embodiment of the present invention, the sivelestat or the pharmaceutically acceptable salt thereof is a sivelestat sodium salt, or is described as sivelestat sodium. Further, the sivelestat sodium can further exist in the form of a hydrate, such as a monohydrate, a dihydrate, a trihydrate, a tetrahydrate. In a preferred embodiment of the present invention, the sivelestat sodium is a sivelestat sodium tetrahydrate.

The “acute pain” in the present invention mainly refers to post-traumatic pain or post-operative pain (or post-operative pain for short), which has a relatively short duration. Trauma can be caused by factors such as sprains, muscle strains, external injuries and tooth extractions. The acute pain can be mild pain, moderate pain or severe pain in terms of pain intensity.

The “chronic pain” in the present invention refers to pain that lasts for a long time (for example, the pain duration exceeds 3 months). It mainly involves various diseases, including nociceptive pain (such as the pain caused by rheumatoid arthritis), neuropathic pain, organ-related pain (such as the pain induced by pancreatitis, inflammatory bowel syndrome, etc.), mixed pain, etc.

The “inflammatory pain” in the present invention refers to pain associated with various inflammatory diseases, such as pain caused by inflammatory diseases such as rheumatoid arthritis, osteoarthritis, ankylosing spondylitis and scapulohumeral periarthritis.

A suitable COX-2 inhibitor in the present invention includes, but is not limited to: parecoxib, celecoxib, etoricoxib, valdecoxib, imrecoxib, lumiracoxib, zaltoprofen, meloxicam, diclofenac potassium, lornoxicam, ketorolac tromethamine, nimesulide, etodolac, polmacoxib, etc.

As a preferred embodiment of the present invention, the COX-2 inhibitor is parecoxib, celecoxib, diclofenac potassium, lornoxicam, ketorolac tromethamine.

As a preferred embodiment of the present invention, the COX-2 inhibitor is parecoxib, celecoxib, lornoxicam, ketorolac tromethamine.

### Dosage Form and Dosage Selection

The sivelestat or the pharmaceutically acceptable salt thereof in the present invention can be adaptively adjusted according to factors such as the body weight, sex, age, and presence or absence of other diseases of the administration subject, such as a human. It can be administered as a single dose, multiple doses or continuous doses. Calculated based on the cumulative daily dosage, a suitable dosage of the sivelestat is 0.1-500 mg/kg/day, preferably 0.5-400 mg/kg/day, further preferably 0.5-300 mg/kg/day, further preferably 1-300 mg/kg/day, further preferably 1-200 mg/kg/day, and further preferably 1-150 mg/kg/day. In a specific embodiment of the present invention, a daily dosage of the sivelestat or the salt thereof can be 1.0 mg/kg/day, 2.0 mg/kg/day, 3.0 mg/kg/day, 4.0 mg/kg/day, 5.0 mg/kg/day, 6.0 mg/kg/day, 7.0 mg/kg/day, 8.0 mg/kg/day, 9.0 mg/kg/day, 10.0 mg/kg/day, 15.0 mg/kg/day, 20.0 mg/kg/day, 25.0 mg/kg/day, 30.0 mg/kg/day, 35.0 mg/kg/day, 40.0 mg/kg/day, 50.0 mg/kg/day, 60.0 mg/kg/day, 70.0 mg/kg/day, 80.0 mg/kg/day, 90.0 mg/kg/day, 100.0 mg/kg/day, 150.0 mg/kg/day, 200.0 mg/kg/day, 250.0 mg/kg/day, 300.0 mg/kg/day.

The COX-2 inhibitor in the present invention can be adaptively adapted according to factors such as the body weight, sex, age, and presence or absence of other diseases of the administration subject, such as a human, and it can be administered in a single-dose, multi-dose, or continuous-dose manner. Calculated based on the cumulative daily dosage, a suitable dosage of the COX-2 inhibitor is 0.1-300 mg/kg/day, preferably 0.2-250 mg/kg/day, further preferably 0.2-200 mg/kg/day, further preferably 0.2-150 mg/kg/day, and further preferably 0.2-100 mg/kg/day. In a specific embodiment of the present invention, a daily dosage of the COX-2 inhibitor can be 0.2 mg/kg/day, 0.3 mg/kg/day, 0.5 mg/kg/day, 1.0 mg/kg/day, 1.5 mg/kg/day, 2.0 mg/kg/day, 3.0 mg/kg/day, 4.0 mg/kg/day, 5.0 mg/kg/day, 6.0 mg/kg/day, 7.0 mg/kg/day, 8.0 mg/kg/day, 9.0 mg/kg/day, 10.0 mg/kg/day, 20.0 mg/kg/day, 30.0 mg/kg/day, 40.0 mg/kg/day, 50.0 mg/kg/day, 60.0 mg/kg/day, 70.0 mg/kg/day, 80.0 mg/kg/day, 90.0 mg/kg/day, 100.0 mg/kg/day, 150.0 mg/kg/day, 200.0 mg/kg/day.

When the sivelestat or the pharmaceutically acceptable salt thereof is used in combination with the COX-2 inhibitor, a ratio of the two is from 100:1 to 1:1 (weight ratio), preferably ranges from 100:1 to 2:1, further preferably ranges from 100:1 to 3:1, and more preferably ranges from 50:1 to 3:1, or from 45:1 to 3:1, or from 40:1 to 3:1, or from 35:1 to 3:1, or from 30:1 to 3:1, or from 25:1 to 3:1, or from 20:1 to 3:1, or from 15:1 to 3:1, or from 10:1 to 3:1.

In a specific embodiment of the present invention, a specific ratio of the sivelestat or the salt thereof to the COX-2 inhibitor can be 100:1, 90:1, 80:1, 70:1, 60:1, 50:1, 45:1, 40:1, 35:1, 30:1, 25:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1.

In one specific embodiment of the present invention, a ratio range of the sivelestat sodium to the parecoxib is from 100:1 to 1:1, preferably from 50:1 to 1:1, and further preferably from 50:1 to 3:1, or 45:1 to 3:1, or 40:1 to 3:1, or 35:1 to 3:1, or 30:1 to 3:1, or 25:1 to 3:1, or 20:1 to 3:1, or 15:1 to 3:1, or 10:1 to 3:1. In a specific embodiment of the present invention, a specific ratio of the sivelestat sodium to the parecoxib can be 50:1, 45:1, 40:1, 35:1, 30:1, 25:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1.

The sivelestat or the pharmaceutically acceptable salt thereof and the COX-2 inhibitor in the present invention can be administered simultaneously, sequentially, or at staggered times. They can be administered before or after a meal, and can be administered continuously or intermittently.

The sivelestat or the pharmaceutically acceptable salt thereof and the COX-2 inhibitor in the present invention can be administered via any conventional routes, such as oral administration, injection administration, inhalation administration, etc.

The sivelestat and the COX-2 inhibitor in the present invention can be prepared into preparations with any pharmaceutically acceptable excipients. These preparations include: oral solid preparations, oral liquid preparations, injections, solid preparations for inhalation, liquid preparations for inhalation, sustained-release preparations, etc. Preferred preparations are solid preparations for inhalation and injections. In one specific embodiment of the present invention, both the sivelestat sodium and the COX-2 inhibitor are freeze-dried preparations for injection.

Pharmaceutically acceptable excipients include bulking agents, pH regulators, preservatives, surfactants, etc. Examples of bulking agents are mannitol, and examples of pH regulators are phosphoric acid, sodium dihydrogen phosphate, disodium hydrogen phosphate, sodium hydroxide, etc.

The following animal experiments are used to further elaborate the inventive concept of the present invention, and should not be construed as any limitation to the present invention. The animals, reagents, sivelestat sodium, COX-2 inhibitors (parecoxib, celecoxib, etc.) used in the examples of the present invention are all commercially available.

### Example 1

The experimental results were expressed as “mean±standard deviation”. The data of each group were statistically analyzed using the SPSS16.0 software package to compare whether there were statistical differences among the groups. P<0.05 indicated a statistical difference.

### Experimental Animals:

A total of 72 SD male rats aged 6 to 8 weeks, with body weights of 188-208 g, were divided into 9 groups, with 8 rats in each group.

### Preparation of Carrageenan-Induced Pain Models

Inducer: carrageenan (3%)
Route: subcutaneous injection into the plantar surface of the left hind paw
Injection volume: 100 μL per animal
Injection frequency: single injection on the 1st day of the experiment
Carrageenan preparation: 600 mg of carrageenan powder was weighed and transferred into a glass bottle. Then 20 mL of normal saline was added to the glass bottle. The glass bottle was placed on a magnetic stirrer, and magnetic stirring was performed for 6 hours to obtain a 3% carrageenan solution. The carrageenan solution was stored at 4° C.

### Administration Method

1 Fasting: no
2 Administration route: IP (intraperitoneal injection)
3 Administration volume: 5 mL/kg
4 Administration frequency: single-dose administration
5 Time of administration: administered immediately after induction

### Preparation of Experimental Samples

Sivelestat sodium: 26 mg of sivelestat sodium (corresponding to a dosage of 10 mg/kg) was weighed. Part of normal saline was added, and sonication and vortexing were performed. The pH was adjusted with 10 mol NaH until a homogeneous solution was obtained, and finally, the volume was made up to 13.0 mL. Experimental samples with other dosages (30 mg/kg, 100 mg/kg) could be prepared according to the specific dosages with reference to a similar method.

Parecoxib: 24 mg of parecoxib (corresponding to a dosage of 10 mg/kg) was weighed. Part of normal saline was added, and sonication and vortexing were performed. The pH was adjusted with 10 mol NaOH until a homogeneous solution was obtained, and finally, the volume was made up to 12.0 mL. Experimental samples with other dosages (20 mg/kg) could be prepared according to the specific dosages with reference to a similar method. Samples of other COX-2 inhibitors were prepared with reference to the similar method described above.

### Administration Mode

| | | | | | Number | Administration |
|---|---|---|---|---|---|---|
| | | Dosage | Administration | | of | volume |
| Group | Compound | (mg/kg) | route | Carrageenan | animals | (mL/kg) |
| 1. | Normal saline | — | Intraperitoneal | Yes | 8 | 5 |
| 2. | Sivelestat sodium | 10 | Intraperitoneal | Yes | 8 | 5 |
| 3. | Sivelestat sodium | 30 | Intraperitoneal | Yes | 8 | 5 |
| 4. | Sivelestat sodium | 100 | Intraperitoneal | Yes | 8 | 5 |
| 5. | Parecoxib | 10 | Intraperitoneal | Yes | 8 | 5 |
| 6. | Parecoxib | 20 | Intraperitoneal | Yes | 8 | 5 |
| 7. | Sivelestat sodium + | 30 + 10 | Intraperitoneal | Yes | 8 | 2.5 + 2.5 |
| | parecoxib | | | | | |
| 8. | Sivelestat sodium + | 100 + 10 | Intraperitoneal | Yes | 8 | 2.5 + 2.5 |
| | parecoxib | | | | | |
| 9. | Sivelestat sodium + | 100 + 10 | Intraperitoneal | Yes | 8 | 2.5 + 2.5 |
| | celecoxib | | | | | |

### Determination of Thermal Radiation

The animals were placed in a transparent plastic box, and could be allowed to acclimatize for 10 minutes before the test. The middle of the plantar surface of the hind limb on the side where carrageenan was injected in the rats was irradiated with a radiant heat light source to determine their thermal pain threshold. The detection was repeated twice. Then the mean value was taken as the final detection index.

The determination of thermal radiation was performed at a total of 5 time points, including before administration and 1 h, 3 h, 4 h and 6 h after administration.

| | Relative value of thermal radiation (%) | | | |
|---|---|---|---|---|
| | 1 hour after | 3 hours after | 4 hours after | 6 hours after |
| Mean ± SD | administration | administration | administration | administration |
| G1: carrageenan + normal | 1.0 ± 26.1 | −0.2 ± 13.6 | −0.4 ± 8.7 | 0.4 ± 11.2 |
| saline | | | | |
| G2: carrageenan + sivelestat | 10.3 ± 28.7 | 19.8 ± 14.0* | 3.7 ± 5.1 | 4.2 ± 6.2 |
| sodium (10 mg/kg) | | | | |
| G3: carrageenan + sivelestat | 20.9 ± 39.4 | 18.0 ± 21.4 | 4.0 ± 9.3 | 9.2 ± 4.4 |
| sodium (30 mg/kg) | | | | |
| G4: carrageenan + sivelestat | 24.0 ± 41.2 | 22.3 ± 18.0* | 13.1 ± 11.0 | 5.4 ± 8.1 |
| sodium (100 mg/kg) | | | | |
| G5: carrageenan + parecoxib | 34.6 ± 18.9* | 16.6 ± 16.7 | 22.7 ± 13.8* | 14.6 ± 11.0* |
| (10 mg/kg) | | | | |
| G6: carrageenan + parecoxib | 45.8 ± 25.1** | 58.5 ± 22.3** | 37.4 ± 26.7 | 38.0 ± 11.0** |
| (20 mg/kg) | | | | |
| G7: carrageenan + sivelestat | 40.1 ± 30.8** | 51.3 ± 20.2** | 39.1 ± 18.4** | 33.1 ± 14.2** |
| sodium (30 mg/kg) + | | | | |
| parecoxib (10 mg/kg) | | | | |
| G8: carrageenan + sivelestat | 50.4 ± 21.6** | 49.3 ± 26.9** | 40.9 ± 11.0** | 39.3 ± 18.9** |
| sodium (100 mg/kg) + | | | | |
| parecoxib (10 mg/kg) | | | | |
| G9: carrageenan + sivelestat | 31.6 ± 14.9* | 13.6 ± 10.7 | 18.7 ± 11.5* | 12.6 ± 8.1* |
| sodium (100 mg/kg) + | | | | |
| celecoxib (10 mg/kg) | | | | |

Relative value of thermal radiation (%) = (detected value − mean value of the first group)/(baseline value before carrageenan modeling − mean value of the first group) * 100. One-way analysis of variance between groups was performed using SPSS software. Compared with the first group, P < 0.05 represented a significant difference, indicated by “*”; and P < 0.01 represented a highly significant difference, indicated by “**”.

In the thermal pain threshold test, sivelestat sodium used alone at dosages of 10 mg/kg and 100 mg/kg had significant analgesic efficacy 3 hours after administration. When combined with low-dosage parecoxib, the analgesic efficacy was significantly higher than that of sivelestat sodium used alone. The combination of sivelestat sodium at 30 mg/kg had a better analgesic effect than the low-dosage parecoxib used alone. The combination of sivelestat sodium at 100 mg/kg had a better analgesic effect than high-dosage parecoxib used alone. Moreover, the duration of drug action was significantly prolonged, and the drug efficacy could still be maintained 6 h after administration.

### Determination of Mechanical Pain Thresholds

The animals were placed in a specially designed multi-unit metal mesh cage for pain detection, and allowed to acclimate to the environment for 10 minutes. After the grooming and exploratory activities of the animals ceased and the animals adapted to the detection environment, the sole of the hind limb on the side where carrageenan was injected in the rats was stimulated with electronic Von Frey Hairs. Pressure was continuously increased until the rats showed an obvious paw-withdrawal response. The value of the Von Frey Hairs at this time, which was the threshold of the mechanical pain response, was recorded with “gram” as the unit of detection. The detection was repeated twice. Then the mean value was taken as the final detection index.

The determination of mechanical pain thresholds was performed at a total of 5 time points, including before administration and 1 h, 3 h, 4 h and 6 h after administration.

| | Relative value of Von Frey Hairs (%) | | | |
|---|---|---|---|---|
| | 1 hour after | 3 hours after | 4 hours after | 6 hours after |
| Mean ± SD | administration | administration | administration | administration |
| G1: carrageenan + normal | 0.5 ± 3.9 | 0.6 ± 4.2 | 0.6 ± 3.2 | −0.1 ± 4.1 |
| saline | | | | |
| G2: carrageenan + sivelestat | 17.1 ± 5.5** | 15.3 ± 5.9** | 11.5 ± 6.1** | 4.8 ± 4.4* |
| sodium (10 mg/kg) | | | | |
| G3: carrageenan + sivelestat | 18.9 ± 5.9** | 15.6 ± 7.5* | 15.2 ± 4.2** | 4.1 ± 5.0 |
| sodium (30 mg/kg) | | | | |
| G4: carrageenan + sivelestat | 20.4 ± 3.9** | 18.1 ± 3.7** | 15.5 ± 4.3** | 7.9 ± 4.5** |
| sodium (100 mg/kg) | | | | |
| G5: carrageenan + parecoxib | 13.6 ± 5.9** | 16.4 ± 4.2** | 15.1 ± 4.5** | 11.9 ± 5.8** |
| (10 mg/kg) | | | | |
| G6: carrageenan + parecoxib | 13.7 ± 7.5** | 17.3 ± 7.9** | 16.2 ± 4.2** | 12.4 ± 3.9** |
| (20 mg/kg) | | | | |
| G7: carrageenan + sivelestat | 14.5 ± 5.2** | 24.2 ± 6.6** | 15.6 ± 6.0** | 13.4 ± 3.6** |
| sodium (30 mg/kg) + | | | | |
| parecoxib (10 mg/kg) | | | | |
| G8: carrageenan + sivelestat | 17.3 ± 4.8** | 24.0 ± 7.1** | 17.7 ± 4.3** | 12.8 ± 3.9** |
| sodium (100 mg/kg) + | | | | |
| parecoxib (10 mg/kg) | | | | |
| G9: carrageenan + sivelestat | 13.2 ± 6.5** | 15.3 ± 4.1** | 12.6 ± 4.6** | 9.8 ± 3.5** |
| sodium (100 mg/kg) + | | | | |
| celecoxib (10 mg/kg) | | | | |

Relative value of Von Frey Hairs (%) = (detected value − mean value of the first group)/(baseline value before carrageenan modeling − mean value of the first group) * 100. One-way analysis of variance between groups was performed using SPSS software. Compared with the first group, P < 0.05 represented a significant difference, indicated by “*”; and P < 0.01 represented a highly significant difference, indicated by “**”

In the rat carrageenan models, sivelestat sodium used alone at dosages of 10, 30 and 100 mg/kg showed significant analgesic efficacy in the mechanical pain threshold test, and there was a certain dosage gradient effect. When sivelestat sodium at 30 mg/kg was combined with low-dosage parecoxib, the drug efficacy was stronger than that of the low-dosage parecoxib used alone. When sivelestat sodium at 100 mg/kg was combined with the low-dosage parecoxib, the drug efficacy was stronger than that of high-dosage parecoxib used alone. Moreover, the drug efficacy could still be maintained 6 hours after administration.

### Determination of Paw Swelling Volumes

To measure the paw swelling volumes (paw volumes) of experimental rats, the animals were gently held with one hand, and their hind paws were placed into the instrument used to measure the paw volumes with the other hand. The size of the paw volume was recorded. The measurement was repeated twice, and the mean value was taken as the paw volume of the animals.

The determination of paw swelling volumes was performed at a total of 5 time points, including before administration and 1 h, 3 h, 4 h and 6 h after administration.

| | Relative value of paw volumes (%) | | | |
|---|---|---|---|---|
| | 1 hour after | 3 hours after | 4 hours after | 6 hours after |
| Mean ± SD | administration | administration | administration | administration |
| G1: carrageenan + normal | −0.43 ± 22.98 | 0.08 ± 18.35 | 0.23 ± 13.67 | −0.07 ± 15.47 |
| saline | | | | |
| G2: carrageenan + sivelestat | 4.86 ± 19.10 | 8.96 ± 13.90 | 1.67 ± 15.59 | 3.70 ± 14.04 |
| sodium (10 mg/kg) | | | | |
| G3: carrageenan + sivelestat | 20.49 ± 41.12 | 17.37 ± 17.14 | 14.09 ± 18.97 | 14.94 ± 18.68 |
| sodium (30 mg/kg) | | | | |
| G4: carrageenan + sivelestat | 3.99 ± 24.23 | 18.24 ± 11.26 | 13.64 ± 15.23 | 19.27 ± 11.40* |
| sodium (100 mg/kg) | | | | |
| G5: carrageenan + parecoxib | 4.34 ± 25.02 | 24.76 ± 11.32* | 22.58 ± 18.82* | 20.81 ± 12.66* |
| (10 mg/kg) | | | | |
| G6: carrageenan + parecoxib | 18.58 ± 45.76 | 31.45 ± 25.56** | 19.47 ± 23.41 | 23.95 ± 15.74* |
| (20 mg/kg) | | | | |
| G7: carrageenan + sivelestat | −8.68 ± 37.54 | 24.92 ± 26.52* | 12.54 ± 33.43 | 15.05 ± 24.14 |
| sodium (30 mg/kg) + | | | | |
| parecoxib (10 mg/kg) | | | | |
| G8: carrageenan + sivelestat | 16.93 ± 32.06 | 32.86 ± 21.99** | 27.88 ± 25.78* | 31.63 ± 23.45** |
| sodium (100 mg/kg) + | | | | |
| parecoxib (10 mg/kg) | | | | |
| G9: carrageenan + sivelestat | 4.124 ± 25.02 | 21.36 ± 10.42* | 19.65 ± 16.32 | 15.45 ± 13.25 |
| sodium (100 mg/kg) + | | | | |
| celecoxib (10 mg/kg) | | | | |

Relative value of paw volumes (%) = (detected value − mean value of the first group)/(baseline value before carrageenan modeling − mean value of the first group) * 100. One-way analysis of variance between groups was performed using SPSS software. Compared with the first group, P < 0.05 represented a significant difference, indicated by “*”; and P < 0.01 represented a highly significant difference, indicated by “**”.

The results of paw volumes showed that sivelestat sodium used alone at dosages of 10, 30 and 100 mg/kg showed a certain anti-inflammatory effect compared with the model group. Sivelestat sodium at 100 mg/kg had a significant anti-inflammatory effect 6 h after administration. The combination of sivelestat sodium at 100 mg/kg and parecoxib at 10 mg/kg showed a significant anti-inflammatory effect at 3, 4 and 6 h after administration. Moreover, the drug efficacy was stronger than that of parecoxib at 20 mg/kg, and a relatively strong drug efficacy could still be maintained 6 h after administration.

The above determination tests of thermal radiation and mechanical pain thresholds showed that the sivelestat sodium in the present invention alone or in combination with the COX-2 inhibitor had a significant therapeutic effect on inflammatory pain. The determination experiments of paw swelling volumes showed that the sivelestat sodium in the present invention alone or in combination with the COX-2 inhibitor also had a certain anti-inflammatory effect.

### Example 2

Drug Efficacy Evaluation of Sivelestat Sodium Combined with Parecoxib in Rat Models of Post-Operative Pain

Male SD rats were acclimated to the test environment for 3 days before modeling. On the day of modeling, the animals were anesthetized with Zoletil 50+xylazine hydrochloride (20 mg/kg+8 mg/kg, intraperitoneal injection). A longitudinal incision of approximately 1 cm in length was made towards the toes at a position 0.7-0.8 cm from the heel of the left hind paw of the animals. After the skin was incised, the flexor digitorum brevis muscle was lifted, and a longitudinal blunt injury was inflicted. On the first day after the operation, the model animals were randomly divided into 7 groups according to the baseline values of mechanical hyperalgesia. The animal experiment protocol is shown in the following table.

| | | | | | Administration | Number | |
|---|---|---|---|---|---|---|---|
| Group | | | Administration | Dosage | volume | of | |
| number | Model | Group | route | (mg/kg) | (mL/kg) | animals | Test time |
| 1 | Post- | Vehicle | i.p. | — | — | 8 | Test at 1, 2 |
| | operative | (normal | | | | | and 6 hours |
| | pain | saline) | | | | | after |
| 2 | Post- | Parecoxib | i.p. | 20 | 5 | 8 | administration |
| | operative | | | | | | |
| | pain | | | | | | |
| 3 | Post- | Sivelestat | i.p. | 10 | 5 | 8 | |
| | operative | sodium | | | | | |
| | pain | | | | | | |
| 4 | Post- | Sivelestat | i.p. | 30 | 5 | 8 | |
| | operative | sodium | | | | | |
| | pain | | | | | | |
| 5 | Post- | Sivelestat | i.p. | 100 | 5 | 8 | |
| | operative | sodium | | | | | |
| | pain | | | | | | |
| 6 | Post- | Sivelestat | i.p. | 30 + 10 | 5 | 8 | |
| | operative | sodium + | | | | | |
| | pain | parecoxib | | | | | |
| 7 | Post- | Sivelestat | i.p. | 100 + 10 | 5 | 8 | |
| | operative | sodium + | | | | | |
| | pain | parecoxib | | | | | |

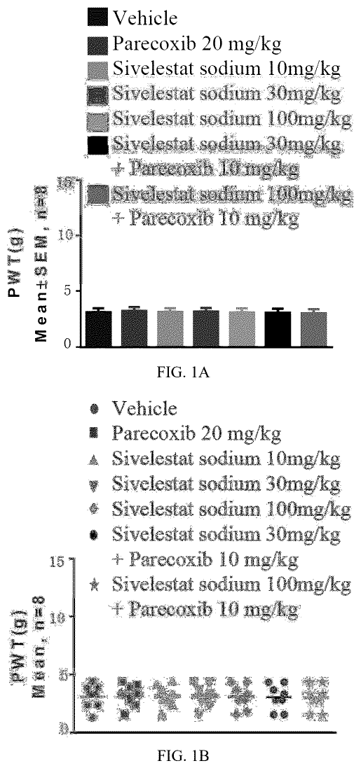

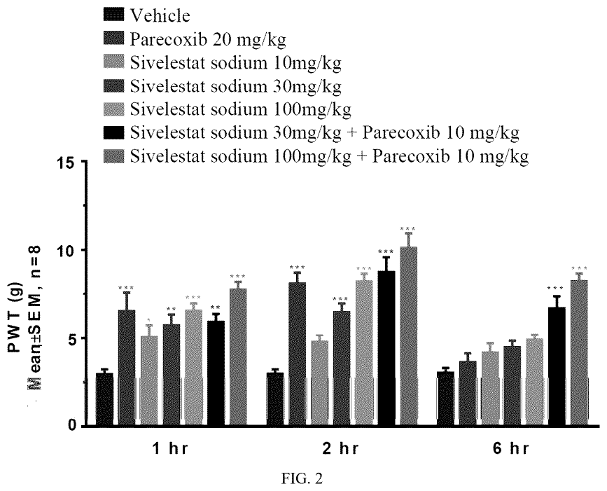

Determination of baseline values: on the first day after the operation (day 1), the baseline values of mechanical hyperalgesia in the experimental rats were determined, and the rats were randomly grouped according to the test results. The experimental results are shown in FIG. 1.
Drug administration and testing: drugs were administered on the first day after the operation (single-dose administration). The mechanical hyperalgesia threshold of the experimental rats was measured at 1, 2 and 6 h after administration, separately. The experimental results are shown in FIGS. 2 and 3. In FIG. 2, “*” indicated p < 0.05, “**” indicated p < 0.01, and “***” indicated p < 0.001, compared with the vehicle control group.

Two-way analysis of variance with the Bonferroni's multiple comparison test was performed.

The mean value of each group of data is shown in the following table.

| | Mechanical hyperalgesia | | | |
|---|---|---|---|---|
| | test (PWT(g)) | | | |
| Grouping | Baseline value | [symbol] | [symbol] | [symbol] |
| Vehicle group | 3.098 | [symbol] | [symbol] | [symbol] |
| Mean value | | | | |
| Parecoxib 20 mg/kg group | 3.215 | [symbol] | [symbol] | [symbol] |
| Mean value | | | | |
| Sivelestat sodium 10 mg/kg group | 3.133 | [symbol] | [symbol] | [symbol] |
| Mean value | | | | |
| Sivelestat sodium 30 mg/kg group | 3.153 | [symbol] | [symbol] | [symbol] |
| Mean value | | | | |
| Sivelestat sodium 100 mg/kg group | 3.086 | [symbol] | [symbol] | [symbol] |
| Mean value | | | | |
| Sivelestat sodium + parecoxib 30 | 3.050 | [symbol] | [symbol] | [symbol] |
| mg/kg + 10 mg/kg group | | | | |
| Mean value | | | | |
| Sivelestat sodium + parecoxib | 3.007 | [symbol] | [symbol] | [symbol] |
| 100 mg/kg + 10 mg/kg group | | | | |
| Mean value | | | | |

[symbol]

indicates data missing or illegible when filed

The data indicated that sivelestat sodium, when use alone or the treatment of post-operative pain, had a significant effect. When parecoxib was combined with sivelestat sodium at a dosage of 10 mg/kg, the duration of drug efficacy action was significantly prolonged. The drug efficacy could still be maintained 6 hours after administration. Moreover, the combination significantly improved the therapeutic effect of parecoxib. When combined with low-dosage sivelestat sodium, the drug efficacy was better than that of high-dosage parecoxib alone at 2 h and 6 h after administration. When combined with high-dosage sivelestat sodium, the drug efficacy was better than that of the high-dosage parecoxib alone at 1 h, 2 h and 6 h after administration.

The above specific examples demonstrated that the sivelestat sodium in the present invention could be used for the treatment of acute pain, such as post-traumatic pain or post-operative pain. The combination of the sivelestat sodium and the COX-2 inhibitor in the present invention could significantly improve the efficacy of the COX-2 inhibitor and reduce the dosage of the COX-2 inhibitor, and could significantly prolong the treatment time. This was conducive to reducing the risk of gastrointestinal adverse events associated with non-steroidal anti-inflammatory drugs.

1. A method for treating or alleviating pain in a subject in need thereof, comprising administering sivelestat or a pharmaceutically acceptable salt thereof.

2. The method according to claim 1, wherein the pain is acute pain or chronic pain; the acute pain comprises post-traumatic pain or post-operative pain; and the chronic pain is inflammatory pain.

3. The method according to claim 1, wherein a dosage of the sivelestat or the pharmaceutically acceptable salt thereof is 1-300 mg/kg/day.

4. A method for treating or alleviating pain in a subject in need thereof, comprising administering sivelestat or a pharmaceutically acceptable salt thereof and a hydrate thereof in combination with a COX-2 inhibitor.

5. The method according to claim 4, wherein the pain is acute pain or chronic pain.

6. The method according to claim 5, wherein the acute pain is post-traumatic pain or post-operative pain; and the chronic pain is inflammatory pain.

7. The method according to claim 4, wherein the COX-2 inhibitor is optionally selected from: parecoxib, celecoxib, etoricoxib, valdecoxib, imrecoxib, lumiracoxib, zaltoprofen, meloxicam, diclofenac potassium, lornoxicam, ketorolac tromethamine, nimesulide, etodolac, polmacoxib.

8. The method according to claim 7, wherein the COX-2 inhibitor is parecoxib, celecoxib, diclofenac potassium, lornoxicam, ketorolac tromethamine.

9. The method according to claim 4, wherein a dosage of the sivelestat or the pharmaceutically acceptable salt thereof is 1.0-300 mg/kg/day.

10. The method according to claim 5, wherein a weight ratio of the sivelestat or the pharmaceutically acceptable salt thereof to the COX-2 inhibitor is from 100:1 to 1:1.

11. A combination product, comprising components of:
(1) sivelestat or a pharmaceutically acceptable salt thereof; and
(2) a COX-2 inhibitor.

12. The combination product according to claim 11, wherein the COX-2 inhibitor is optionally selected from: parecoxib, celecoxib, etoricoxib, valdecoxib, imrecoxib, lumiracoxib, zaltoprofen, meloxicam, diclofenac potassium, lornoxicam, ketorolac tromethamine, nimesulide, etodolac, polmacoxib.

13. A method for treating or alleviating pain in a subject in need thereof, comprising administering the combination product according to claim 11.

14. The method according to claim 13, wherein the pain is acute pain or chronic pain; the acute pain comprises post-traumatic pain or post-operative pain; and the chronic pain is inflammatory pain.

15. The combination product according to claim 11, wherein a ratio of the sivelestat or the pharmaceutically acceptable salt thereof to the COX-2 inhibitor is from 100:1 to 1:1.

16. A pharmaceutical kit, comprising the combination product according to claim 11.
